(19) United States
(12) Patent Application Publication
(10) Pub. No.: US 2010/0043106 A1
(43) Pub. Date: Feb. 18, 2010

(54) PTILOTUS

(75) Inventor: Gerald Raser, Wien (AT)

Correspondence Address: JONDLE & ASSOCIATES P.C., 858 HAPPY CANYON ROAD SUITE 230, CASTLE ROCK, CO, 80108, US

(73) Assignee: ERNST BENARY SAMENZUCHT GMBH, Hannoversch Munden (DE)

(21) Appl. No.: 12/603,729

(22) Filed: Oct. 22, 2009

Related U.S. Application Data
(63) Continuation-in-part of application No. 12/324,966, filed on Nov. 28, 2008.
(60) Provisional application No. 60/991,631, filed on Nov. 30, 2007.

(51) Int. Cl. A01H 5/00

(52) U.S. Cl. 800/298

(57) ABSTRACT

A Ptilotus plant with an increased germination rate of 61% or greater, a compact growth habit and decreased number of days to flower is disclosed.

### CROSS-REFERENCE

This application is a continuation-in-part application of U.S. application Ser. No. 12/324,966, filed on Nov. 28, 2008, which claims the benefit of priority to U.S. Provisional Application No. 60/991,631 filed on Nov. 30, 2007, both of which are herein incorporated by reference.

### BACKGROUND

The present invention relates to Ptilotus plants having a high seed germination rate, improved plant uniformity, compact habit and decreased days to flower. All publications cited in this application are herein incorporated by reference.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include increased number of flowers, resistance to diseases and insects, better stems and roots, tolerance to drought and heat, and better horticultural quality.

Choice of breeding or selection methods depends on the mode of plant reproduction, the heritability of the trait(s) being improved, and the type of plant used commercially (e.g., F₁ hybrid variety, pureline variety, etc.). For highly heritable traits, a choice of superior individual plants evaluated at a single location will be effective, whereas for traits with low heritability, selection should be based on mean values obtained from replicated evaluations of families of related plants. Popular selection methods include pedigree selection, modified pedigree selection, mass selection, and recurrent selection.

The complexity of inheritance influences choice of the breeding method. Backcross breeding is used to transfer one or a few favorable genes for a highly heritable trait into a desirable variety. This approach has been used extensively for breeding disease-resistant varieties. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful varieties produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial varieties; those still deficient in a few traits may be used as parents to produce new populations for further selection.

These processes, which lead to the final step of marketing and distribution, usually take from eight to twelve or more years from the time the first cross is made. Therefore, development of new varieties is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of Ptilotus plant breeding is to develop new, unique and superior Ptilotus varieties and hybrids. The breeder initially selects and crosses two or more parental lines, followed by repeated selfing and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same Ptilotus traits.

Each growing season, the plant breeder selects the germplasm to advance to the next generation. This germplasm is grown under unique and different geographical, climatic and soil conditions and further selections are then made, during and at the end of the growing season. The varieties which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same variety twice by using the exact same original parents and the same selection techniques. This unpredictability results in the expenditure of large amounts of research monies to develop superior new plant varieties.

Pedigree breeding and recurrent selection breeding methods are used to develop varieties from breeding populations. Breeding programs combine desirable traits from two or more varieties or various broad-based sources into breeding pools from which varieties are developed by selfing and selection of desired phenotypes. The new varieties are evaluated to determine which have commercial potential. Pedigree breeding is used commonly for the improvement of self-pollinating crops. Two parents which possess favorable, complementary traits are crossed to produce an F₁. An F₂ population is produced by selfing one or several F₁s. Selection of the best individuals may then begin in the F₂ population.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous variety or inbred line which is the recurrent parent. The source of the trait to be transferred is called the donor parent. The resulting plant is expected to have the attributes of the recurrent parent (e.g., variety) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., variety) and the desirable trait transferred from the donor parent.

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen, (Molecular Linkage Map of Soybean (Glycine max L. Merr.) pp. 6.131-6.138 in S. J. O'Brien (ed) Genetic Maps: Locus Maps of Complex Genomes, Cold Spring Harbor Laboratory Press, Cold Spring Harbor, N.Y. (1993)) developed a molecular genetic linkage map that consisted of 25 linkage groups with about 365 RFLP, 11 RAPD, three classical markers and four isozyme loci. See also, Shoemaker, R. C., RFLP Map of Soybean, pp. 299-309, in Phillips, R. L. and Vasil, I. K., eds., DNA-Based Markers in Plants, Kluwer Academic Press, Dordrecht, the Netherlands (1994).

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus with as many as 26 alleles. (Diwan, N. and Cregan, P. B., Theor. Appl. Genet. 95:22-225, 1997.) SNPs may also be used to identify the unique genetic composition of the invention and progeny varieties retaining that unique genetic composition. Various molecular marker techniques may be used in combination to enhance overall resolution.

Molecular markers, which includes markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. For example, molecular markers are used in soybean breeding for selection of the trait of resistance to soybean cyst nematode, see U.S. Pat. No. 6,162,967. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Molecular markers may also be used to identify and exclude certain sources of germplasm as parental varieties or ancestors of a plant by providing a means of tracking genetic profiles through crosses.

Mutation breeding is another method of introducing new traits into canola varieties. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogues like 5-bromo-uracil), antibiotics, alkylating agents (such as sulfur mustards, nitrogen mustards, epoxides, ethyleneamines, sulfates, sulfonates, sulfones, or lactones), azide, hydroxylamine, nitrous acid or acridines. Once a desired trait is observed through mutagenesis the trait may then be incorporated into existing germplasm by traditional breeding techniques. Details of mutation breeding can be found in Principles of Cultivar Development by Fehr, Macmillan Publishing Company, 1993.

Descriptions of other breeding methods that are commonly used for different traits and crops can be found in one of several reference books (e.g., Allard, 1960; Simmonds, 1979; Sneep et al., 1979; Fehr, 1987).

Proper testing should detect any major faults and establish the level of superiority or improvement over current varieties. In addition to showing superior performance, there must be a demand for a new variety that is compatible with industry standards or which creates a new market. The introduction of a new variety will incur additional costs to the seed producer, the grower, processor and consumer for special advertising and marketing, altered seed and commercial production practices, and new product utilization. The testing preceding release of a new variety should take into consideration research and development costs as well as technical superiority of the final variety. For seed-propagated varieties, it must be feasible to produce seed easily and economically.

The genus Ptilotus (family Amaranthaceae) encompasses about 100 recognized species mostly endemic to Australia. Most occur in the arid and semi-arid regions. These plants have diverse morphological characters and growth forms from prostrate to erect herbs and small woody shrubs. The inflorescence is a spike with developing flowers (florets) that gradually open sequentially towards the apex. The profuse flowering of Ptilotus and the long vase life of the harvested flowers has been noted (Williams et al. 1994. Progress with propagation and floral biology of Ptilotus species. In ‘Third National workshop for Australian native flowers’, pp. 4-14 to 4-17. Univ. Queensland, Gatton College; Growns et al. 1996. Developments in Mulla Mulla. In ‘IV National workshop for Australian native flowers, Programme and Proceedings’, pp. 241-245. Univ. Western Australia Press, Perth; Williams. 1996. Ptilotus (Mulla Mulla), Family Amaranthaceae. In ‘Native Australian plants horticulture and uses’, Eds. Johnson, K. A. and M. Burchett. Univ. New South Wales Press, Sydney). Robert Brown (1810) was the first person to describe the genus Ptilotus.

According to Benl (The genus Ptilotus R. Br. Australian Plants. June 1967 pp. 109-117), the first description of the genus Ptilotus was published in 1810. Ptilotus is still not well known today. Statements found in Australian literature indicate an early interest in the use of the Ptilotus species as garden plants, as early as 1845 (Benl, G., 1971. Ein Bestimmungsschluessel fuer die Gattung Ptilotus R. Br. Amaranthaceae. Mitteilungen der Botanischen Staatssammlung Muenchen. Bd.IX:135-176). The development of Ptilotus into a garden plant has been problematic.

Among the problems encountered in attempting to develop Ptilotus as a garden plant are difficulties in germinating the seeds and lack of uniformity among the plants produced (Benl, G., 1967). Most of the experimentation with Ptilotus for developing garden plants has involved the species Ptilotus exaltatus Nees (Williams et al., 1989. Cultivation of the pink mulla mulla Ptilotus exaltatus Nees. 1. Seed germination and dormancy. Scientia Hort. 40:267-274; Williams et al., 1990. Propagation of Ptilotus exaltatus. Australia Hort. February 83-84; Bennel et al., 1992. Cultivation of the pink mulla mulla, Ptilotus exaltatus Nees. 2. Nutrition and growth regulation. Scientia Hort. 51:107-110). Efforts to improve seed germination have required lengthy cold storage of the seeds followed by treatment with gibberelic acid. Efforts to improve uniformity and growth habit have required pinching and treatment with growth regulators such as chlormequat-chlorid/cholinchlorid. (See Hentig, W. U. et al., 1995. The development of Ptilotus exaltatus R. Br. under central European conditions. Acta Hort. 397:163-180.)

Storing seeds for long periods of time and treating seeds and plants with plant growth regulators are expensive and labor intensive. For Ptilotus to be successful as a garden plant, whether in pots or in a garden bed, it would be desirable to have Ptilotus seeds which germinated readily and at a high rate without the use of gibberelic acid or other plant growth regulators. It would also be desirable to have Ptilotus plants which were of compact size and uniform shape and growth habit without the need for pinching the plants or applying plant growth regulators.

### SUMMARY OF THE INVENTION

The following embodiments and aspects thereof are described in conjunction with systems, tools and methods which are meant to be exemplary and illustrative, not limiting in scope. In various embodiments, one or more of the above-described problems have been reduced or eliminated, while other embodiments are directed to other improvements.

According to the invention, there is provided a new Ptilotus plant with increased germination rates, improved plant uniformity and compact plant habit and decreased days to flower.

In another aspect, the present invention provides a new Ptilotus plant with increased germination rates of 61%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100.0% inclusive.

In another aspect, the present invention provides a Ptilotus plant with a plant height grown without a growth regulator between 350.0 mm and 450.0 mm.

In another aspect, the present invention provides a Ptilotus plant with a plant height grown with a growth regulator between 250.0 mm and 310.0 mm.

In another aspect, the present invention provides a Ptilotus plant that has a number of days to flower of 75 day to 100 days when grown without a grown regulator.

In another aspect, the present invention provides a Ptilotus plant that has a number of days to flower of 80 day to 105 days when grown with a grown regulator.

In addition to the exemplary aspects and embodiments described above, further aspects and embodiments will become apparent by study of the following descriptions.

### DEFINITIONS

In the description and tables that follow, a number of terms are used. In order to provide a clear and consistent understanding of the specification and claims, including the scope to be given such terms, the following definitions are provided:

Allele. An allele is any of one or more alternative forms of a gene which relate to one trait or characteristic. In a diploid cell or organism, the two alleles of a given gene occupy corresponding loci on a pair of homologous chromosomes.

Alter. The utilization of up-regulation, down-regulation, or gene silencing.

Backcrossing. Backcrossing is a process in which a breeder repeatedly crosses hybrid progeny back to one of the parents, for example, a first generation hybrid F₁ with one of the parental genotypes of the F₁ hybrid.

Cell. Cell as used herein includes a plant cell, whether isolated, in tissue culture or incorporated in a plant or plant part.

Compact habit. Means a plant that has a characteristic shape that is shorter and shows a denser habit compared to wild Ptilotus lines due to shorter internodes.

Cotyledon. A cotyledon is one of the first leaves to appear after germination. It is the foliar portion of the embryo as found in the seed.

Days to Flower. Means the number of days from sowing date to first of flowering date.

Embryo. The embryo is the small plant contained within a mature seed.

Gene Silencing. The interruption or suppression of the expression of a gene at the level of transcription or translation.

Genotype. Refers to the genetic constitution of a cell or organism.

Germination. The process where a seed, spore, or zygote begins to sprout, grow, or develop.

Growth Regulator. As used herein, a growth regulator is a chemical or chemicals used to alter the growth of a plant or plant part. A plant grown with a growth regulator will have a chemical or chemicals applied during the development of the plant to alter the growth of the plant or a plant part. A plant grown without a growth regulator will not have a chemical or chemicals applied during the development of the plant to alter the growth of the plant or a plant part.

Habit. Means the characteristic shape or form of a plant.

Hypocotyl. A hypocotyl is the portion of an embryo or seedling between the cotyledons and the root. Therefore, it can be considered a transition zone between shoot and root.

Linkage. Refers to a phenomenon wherein alleles on the same chromosome tend to segregate together more often than expected by chance if their transmission was independent.

Linkage Disequilibrium. Refers to a phenomenon wherein alleles tend to remain together in linkage groups when segregating from parents to offspring, with a greater frequency than expected from their individual frequencies.

Natural Germination. Germination made without the use of artificial, chemical or mechanical techniques.

Natural Germination Rate. Percentage of seed, spore, or zygote sprouting, growing or developing without the use of artificial, chemical or mechanical compounds or techniques.

Pedigree Distance. Relationship among generations based on their ancestral links as evidenced in pedigrees. Genetic similarity decreases with pedigree distance. May be measured by the distance of the pedigree from a given starting point in the ancestry.

Percent Identity. Percent identity as used herein refers to the comparison of the alleles of two Ptilotus varieties. Percent identity is determined by comparing a statistically significant number of the alleles of two developed varieties. For example, a percent identity of 90% between Ptilotus variety 1 and Ptilotus variety 2 means that the two varieties have the same allele at 90% of their loci.

Percent Similarity. Percent similarity as used herein refers to the comparison of the homozygous alleles of a Ptilotus variety such as the present invention with another plant, and if the homozygous allele of the present invention matches at least one of the alleles from the other plant then they are scored as similar. Percent similarity is determined by comparing a statistically significant number of loci and recording the number of loci with similar alleles as a percentage. A percent similarity of 90% between the present invention and another plant means that the present invention matches at least one of the alleles of the other plant at 90% of the loci.

Plant. As used herein, the term “plant” includes reference to an immature or mature whole plant, including a plant from which seed or anthers have been removed. Seed or embryo that will produce the plant is also considered to be the plant.

Plant Diameter. Plant diameter is measured by measuring the distance between the two farthest points on the side of the plant.

Plant Height. Plant height is taken from the top of the soil to the top of the plant and is measured in millimeters. (In Ptilotus, the top of the plant is the terminal flower of the inflorescence at time of flowering. Later on, single side branches can overgrow the inflorescence.)

Plant Parts. As used herein, the term “plant parts” (or a Ptilotus plant, or a part thereof) includes protoplasts, leaves, stems, roots, root tips, anthers, seed, embryo, pollen, ovules, cotyledon, hypocotyl, flower, shoot, tissue, petiole, cells, meristematic cells and the like.

Plant Uniformity. Means a plant habit that is limited in variation.

Quantitative Trait. As used herein, quantitative trait refers to the inheritance of a phenotypic characteristic that varies in degree and can be attributed to the interactions between two or more genes and their environment. Though not necessarily genes themselves, quantitative trait loci (QTLs) are stretches of DNA that are closely linked to the genes that underlie the trait in question. QTLs can be molecularly identified (for example, with PCR or AFLP) to help map regions of the genome that contain genes involved in specifying a quantitative trait. This can be an early step in identifying and sequencing these genes.

Regeneration. Regeneration refers to the development of a plant from tissue culture.

Single Gene Converted (Conversion). Single gene converted (conversion) plants refers to plants which are developed by a plant breeding technique called backcrossing wherein essentially all of the desired morphological and physiological characteristics of a variety are recovered in addition to the single gene transferred into the variety via the backcrossing technique or via genetic engineering.

### DETAILED DESCRIPTION OF THE INVENTION

Ptilotus exaltatus originated in Australia and is generally found in dry, warm climates but can also be found in tropical areas. P. exaltatus is difficult to grow due to its low germination rate. Furthermore, once the plant does begin to grow, the finished product can vary widely in plant height and habit. The present invention is unique in that it provides P. exaltatus plants with increased germination rates.

Ptilotus exaltatus plants, when mature, generally have light green to blue-green leaves, sometimes with reddish tones, and up to 30 cm long, large purple-to-mauve-colored cylindrically-shaped flower spikes.

The present invention encompasses a Ptilotus plant with a germination rate of 61%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100.0% and including all integers and fractions thereof.

The present invention provides a Ptilotus plant with a compact growth when grown without a growth regulator of 345 mm, 358 mm, 369 mm, 381 mm, 392 mm, 396 mm, 408 mm, 413 mm, 420 mm and 425 mm and including all integers and fractions thereof.

The present invention provides a Ptilotus plant with a compact growth when grown with a growth regulator of 245 mm, 248 mm, 255 mm, 268 mm, 273 mm, 280 mm, 292 mm, 301 mm, 312 mm and 315 mm and including all integers and fractions thereof.

The present invention provides a Ptilotus plant with a decrease in days to flower when grown without a growth regulator of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 40 days, 45 days, 50 days, 55 days, 62 days, 73 days, 85 days, 105 days and including all integers and fractions thereof.

The present invention provides a Ptilotus plant with a decrease in days to flower when grown with a growth regulator of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 25 days, 30 days, 40 days, 45 days, 50 days, 55 days, 62 days, 73 days, 85 days, 110 days and including all integers and fractions thereof.

The present invention differs from typical Ptilotus exaltatus by having a high seed germination rate without a special seed treatment such as GA3 application. It also provides a Ptilotus exaltatus plant with a more compact growth habit and uniform growth habit.

The present invention has the following morphologic and other characteristics (based primarily on data collected in Cobbitty, Australia). Please note that all morphological and other characteristics may be influenced by cultural practices such as varying amounts of fertilizers and from trial sites.

### Example 1

### Increase Germination Rate of the Present Invention

Table 1 shows the increased germination rate of the present invention where four repetitions of 200 seeds were each sown of each cultivar, Invention, the Ptilotus of the present invention and four Ptilotus populations. Column one shows the name and the repetition number of the population tested; column two shows the number of plants germinated fourteen days from sowing; column three shows the number of transplantable plugs (plants that are of a size that is easily transplanted) thirty five days from sowing; column four shows the percentage of plants germinated fourteen days after sowing; column five shows the percentage of plants germinated 35 days after sowing.

TABLE 1
Increased Germination Rate of Present Invention
14 and 35 Days After Sowing

| | No. of | | | |
|---|---|---|---|---|
| | Germinated | | Germination | Germination |
| Name of | plants | Transplantable | percentage | percentage |
| population | 14 days after | plugs 35 days | 14 days | 35 days |
| and rep. # | sowing | after sowing | from sowing | from sowing |
| Invention 1 | 166 | 163 | 83 | 81.5 |
| Invention 2 | 163 | 157 | 81.5 | 78.5 |
| Invention 3 | 168 | 161 | 84 | 84 |
| Invention 4 | 171 | 165 | 85.5 | 82.5 |
| Population | 0 | 0 | 0 | 0 |
| A1 | | | | |
| Population | 0 | 0 | 0 | 0 |
| A2 | | | | |
| Population | 0 | 0 | 0 | 0 |
| A3 | | | | |
| Population | 1 | 1 | 0.5 | 0.5 |
| A4 | | | | |
| Population | 8 | 7 | 4 | 3.5 |
| B1 | | | | |
| Population | 9 | 6 | 4.5 | 3 |
| B2 | | | | |
| Population | 3 | 2 | 1.5 | 1 |
| B3 | | | | |
| Population | 4 | 4 | 2 | 2 |
| B4 | | | | |
| Population | 17 | 16 | 8.5 | 8 |
| C1 | | | | |
| Population | 25 | 21 | 12.5 | 10.5 |
| C2 | | | | |
| Population | 23 | 20 | 11.5 | 10 |
| C3 | | | | |
| Population | 20 | 19 | 10 | 9.5 |
| C4 | | | | |

Table 2 shows the germination rate of the Ptilotus of the present invention when compared to Ptilotus populations from Australia after 14 days from sowing the seed and 21 days from sowing the seed. The 100 seeds per sample were sown without GA3 treatment on Jan. 17, 2008 in a greenhouse in Münden, Germany. Please note that the Ptilotus population used in the trials in Germany shown in Table 2 are not identical to the Ptilotus populations used in the trials from Australia as shown in Tables 4, 5, 6 and 7. In Table 2, column one shows the total percentage of usable plants of the present invention 14 days from sowing the seed. Column two shows the total percentage of usable plants of the present invention 21 days from sowing the seed. Column three shows the total percentage of uniform plants of the present invention 14 days from sowing the seed. Column four shows the total percentage of uniform plants of the present invention 21 days from sowing the seed. Column five shows the total percentage of usable plants of the Ptilotus populations 14 days from sowing the seed. Column six shows the total percentage of usable plants of the Ptilotus populations 21 days from sowing the seed. Column seven shows the total percentage of uniform plants of the Ptilotus populations 14 days from sowing the seed. Column eight shows the total percentage of uniform plants of the Ptilotus populations 21 days from sowing the seed.

TABLE 2
Percentage of Usable and Uniform Plants 14 days and 21 days from sowing of seed

| | | | | Ptilotus | Ptilotus | Ptilotus | Ptilotus |
|---|---|---|---|---|---|---|---|
| Invention: | Invention: | Invention: | Invention: | populations: | populations: | populations: | populations: |
| Total | Total | Total | Total | Total | Total | Total | Total |
| percentage | percentage | percentage | percentage | percentage | percentage | percentage | percentage |
| usable | usable | uniform | uniform | usable | usable | uniform | uniform |
| plants 14 | plants 21 | plants 14 | plants 21 | plants 14 | plants 21 | plants 14 | plants 21 |
| days | days | days | days | days | days | days | days |
| 80 | 85 | 77 | 84 | 49 | 56 | 39 | 45 |
| 76 | 77 | 73 | 76 | 47 | 50 | 37 | 38 |
| 81 | 84 | 77 | 83 | 45 | 56 | 39 | 42 |
| 83 | 84 | 79 | 81 | 45 | 53 | 36 | 40 |
| Average | Average | Average | Average | Average | Average | Average | Average |
| 80.0 | 82.5 | 76.5 | 81.0 | 46.5 | 53.8 | 37.8 | 41.3 |

### Example 2

### Varietal Description of a Ptilotus Plant of the Present Invention

Table 3 below shows an example of the varietal description information of a Ptilotus plant of the present invention and is based primarily on data collected at Münden, Germany.

TABLE 3
VARIETY DESCRIPTION INFORMATION

Plant:
Height:
10 weeks from sowing: 30 cm to 40 cm with an inflorescence 5 cm
in length
15 weeks from sowing: 40 cm tall
Diameter: 20.0 cm to 35.0 cm
Leaves:
Length (of largest leaf): 9.0 cm to 12.9 cm
Width (of largest leaf): 3.1 cm to 5.7 cm
Texture: Half succulent foliage
Color: Light silver green
Flowers:
General: Silver-gray color with pink pilose hairs on the edge and tip
Inflorescence:
Number: Between 4 and 11
Length (of first inflorescence): 7.1 cm to 12.2 cm
Diameter (of first inflorescence): 3.0 cm to 3.9 cm

### Example 3

### Compact Height of Present Invention without a Growth Regulator

In an example of the present invention, the cultivar of the present invention was grown without a growth regulator and compared with three Ptilotus populations. The plants were first grown in plug trays in an unheated poly house. The plants were then transplanted to 10 cm pots and placed outside of the poly house.

Table 4 shows the total height (mm) of the present invention when compared with three Ptilotus populations from Australia. Table 4 shows the replication number (column one), the total height of the Ptilotus of the present invention in millimeters (column two), the total height of Ptilotus population A in millimeters (column three), the total height of Ptilotus population B in millimeters (column four) and the total height of Ptilotus population C in millimeters (column five). Sowing of seed was conducted in September 2008 in Macquarie Fields, Australia. The plants were transplanted to 10 cm pots at six weeks and placed outside of the poly house.

TABLE 4
Total plant height without a growth regulator in millimeters

| | Height | Height in mm - | Height in mm - | Hight in mm - |
|---|---|---|---|---|
| | in mm - | Ptilotus | Ptilotus | Ptilotus |
| Plant No. | BEPT107 | population A | population B | population C |
| 1 | 395 | 520 | 435 | 582 |
| 2 | 405 | 532 | 444 | 585 |
| 3 | 390 | 511 | 467 | 587 |
| 4 | 390 | 523 | 453 | 589 |
| 5 | 405 | 561 | 469 | 600 |
| 6 | 395 | 515 | 470 | 590 |
| 7 | 395 | 517 | 489 | 592 |
| 8 | 390 | 521 | 501 | 592 |
| 9 | 385 | 505 | 459 | 603 |
| 10 | 388 | 511 | 465 | 597 |
| 11 | 390 | 513 | 478 | 592 |
| 12 | 390 | 502 | 456 | 590 |
| 13 | 395 | 497 | 433 | 592 |
| 14 | 395 | 503 | 547 | 594 |
| 15 | 385 | 480 | 523 | 612 |
| 16 | 393 | 509 | 471 | 594 |
| 17 | 398 | 507 | 490 | 608 |
| 18 | 397 | 503 | 517 | 602 |
| 19 | 390 | 501 | 490 | 594 |
| 20 | 392 | 476 | 495 | 594 |
| 21 | 396 | 517 | 483 | 594 |
| 22 | 387 | 519 | 481 | 596 |
| 23 | 385 | 520 | 454 | 584 |
| 24 | 390 | 500 | 458 | 604 |
| 25 | 391 | 517 | 510 | 605 |
| 26 | 395 | 519 | 500 | 612 |
| 27 | 392 | 461 | 496 | 596 |
| 28 | 390 | 519 | 487 | 598 |
| 29 | 390 | 501 | 486 | 593 |
| 30 | 390 | 507 | 495 | 594 |
| 31 | 395 | 503 | 493 | 599 |
| 32 | 397 | 493 | 485 | 600 |
| 33 | 390 | 498 | 486 | 596 |
| 34 | 394 | 507 | 480 | 600 |
| 35 | 388 | 503 | 491 | 613 |
| 36 | 390 | 519 | 485 | 600 |
| 37 | 388 | 516 | 452 | 601 |
| 38 | 386 | 510 | 498 | 597 |
| 39 | 389 | 581 | 500 | 600 |
| 40 | 390 | 535 | 482 | 605 |
| 41 | 390 | 524 | 467 | 600 |
| 42 | 390 | 512 | 489 | 589 |
| 43 | 390 | 501 | 465 | 601 |
| 44 | 390 | 503 | 469 | 602 |
| 45 | 388 | 499 | 463 | 610 |
| 46 | 365 | 496 | 486 | 604 |
| 47 | 390 | 519 | 478 | 602 |
| 48 | 385 | 521 | 493 | 596 |
| 49 | 387 | 527 | 462 | 602 |
| 50 | 390 | 510 | 439 | 581 |
| 51 | 392 | 516 | 472 | 593 |
| 52 | 394 | 513 | 437 | 595 |
| 53 | 390 | 545 | 453 | 602 |
| 54 | 388 | 528 | 462 | 590 |
| 55 | 390 | 525 | 484 | 604 |
| 56 | 394 | 505 | 459 | 589 |
| 57 | 392 | 512 | 483 | 604 |
| 58 | 391 | 517 | 476 | 598 |
| 59 | 394 | 519 | 459 | 591 |
| 60 | 396 | 524 | 462 | 610 |
| 61 | 394 | 526 | 518 | 615 |
| 62 | 390 | 457 | 497 | 612 |
| 63 | 390 | 489 | 486 | 609 |
| 64 | 390 | 523 | 500 | 604 |
| 65 | 392 | 532 | 476 | 608 |
| 66 | 398 | 541 | 485 | 598 |
| 67 | 390 | 500 | 592 | 612 |
| 68 | 388 | 485 | 596 | 615 |
| 69 | 392 | 496 | 481 | 604 |
| 70 | 394 | 527 | 482 | 602 |
| 71 | 388 | 498 | 507 | 609 |
| 72 | 390 | 482 | 480 | 614 |
| 73 | 394 | 513 | 495 | 618 |
| 74 | 390 | 521 | 486 | 610 |
| 75 | 397 | 567 | 495 | 620 |
| 76 | 388 | 483 | 490 | 613 |
| 77 | 387 | 514 | 507 | 614 |
| 78 | 379 | 602 | 486 | 614 |
| 79 | 388 | 498 | 496 | 621 |
| 80 | 394 | 531 | 489 | 617 |
| 81 | 378 | 503 | 490 | 619 |
| 82 | 390 | 507 | 513 | 598 |
| 83 | 388 | 478 | 517 | 614 |
| 84 | 394 | 523 | 498 | 618 |
| 85 | 396 | 461 | 531 | 615 |
| 86 | 394 | 503 | 523 | 613 |
| 87 | 387 | 507 | 531 | 608 |
| 88 | 390 | 512 | 509 | 612 |
| 89 | 393 | 517 | 511 | 604 |
| 90 | 395 | 551 | 487 | 623 |
| 91 | 388 | 497 | 478 | 621 |
| 92 | 390 | 489 | 508 | 608 |
| 93 | 394 | 507 | 524 | 604 |
| 94 | 390 | 504 | 507 | 602 |
| 95 | 393 | 479 | 498 | 606 |
| 96 | 394 | 509 | 476 | 624 |
| 97 | 390 | 513 | 437 | 582 |
| 98 | 388 | 512 | 533 | 608 |
| 99 | 390 | 498 | 487 | 612 |
| 100 | 390 | 491 | 527 | 600 |
| Average | 390.87 | 511.14 | 487.41 | 602.59 |
| height | | | | |

As can be seen in Table 4, the Ptilotus of the present invention is unexpectedly shorter and more compact in height than the three Ptilotus comparison populations. The average height of the present invention was 390.87 mm while the average height of the three Ptilotus populations was between 487.41 mm and 602.59 mm.

### Example 4

### Ptilotus Height with a Growth Regulator

In an example of the novelty of the present invention, the cultivar of the present invention was grown without a growth regulator and compared with three Ptilotus populations. The plants were first grown in plug trays in an unheated poly house. The plants were then transplanted to 10 cm pots and placed outside of the poly house. The growth regulator Regalis, with an active substance of Prohexadion-Ca, was applied twice to the plants at a concentration of 0.5 g/litre. The Regalis was applied as a drench on the plug trays at five weeks, which was one week prior to planting in the pots. The second application was applied two weeks after potting.

Table 5 shows the total height (mm) of the present invention when grown with a growth regulator when compared with three Ptilotus populations from Australia. Table 5 shows the replication number (column one), the total height in millimeters of the Ptilotus of the present invention when grown with a growth regulator (column two), the total height in millimeters of Ptilotus population A (column three), the total height in millimeters of Ptilotus population B (column four) and the total height in millimeters of Ptilotus population C (column five). Sowing of seed was conducted in September 2008 in Macquarie Fields, Australia. The plants were transplanted to 10 cm pots at six weeks and placed outside of the poly house.

TABLE 5
Total plant height with a growth regulator in millimeters

| | Height | Height in mm | Height in mm | Height in mm |
|---|---|---|---|---|
| | in mm | with growth | with growth | with growth |
| | with growth | regulator - | regulator - | regulator - |
| Plant | regulator - | Ptilotus | Ptilotus | Ptilotus |
| No. | BEPT107 | population A | population B | population C |
| 1 | 298 | 501 | 403 | 478 |
| 2 | 284 | 503 | 412 | 464 |
| 3 | 287 | 478 | 415 | 464 |
| 4 | 290 | 456 | 402 | 470 |
| 5 | 290 | 389 | 395 | 472 |
| 6 | 288 | 436 | 424 | 474 |
| 7 | 286 | 471 | 425 | 465 |
| 8 | 287 | 421 | 434 | 467 |
| 9 | 290 | 500 | 401 | 470 |
| 10 | 292 | 471 | 421 | 480 |
| 11 | 294 | 462 | 420 | 465 |
| 12 | 296 | 465 | 413 | 461 |
| 13 | 290 | 432 | 425 | 476 |
| 14 | 288 | 411 | 427 | 484 |
| 15 | 288 | 423 | 432 | 475 |
| 16 | 292 | 420 | 437 | 468 |
| 17 | 293 | 434 | 438 | 464 |
| 18 | 287 | 431 | 461 | 463 |
| 19 | 288 | 435 | 431 | 484 |
| 20 | 290 | 437 | 432 | 459 |
| 21 | 294 | 505 | 430 | 468 |
| 22 | 293 | 487 | 435 | 466 |
| 23 | 290 | 475 | 437 | 470 |
| 24 | 289 | 454 | 434 | 474 |
| 25 | 268 | 563 | 451 | 472 |
| 26 | 280 | 435 | 423 | 468 |
| 27 | 284 | 437 | 428 | 466 |
| 28 | 293 | 427 | 439 | 463 |
| 29 | 286 | 429 | 437 | 462 |
| 30 | 292 | 431 | 440 | 468 |
| 31 | 288 | 457 | 426 | 478 |
| 32 | 287 | 468 | 438 | 476 |
| 33 | 290 | 423 | 463 | 467 |
| 34 | 293 | 430 | 441 | 483 |
| 35 | 286 | 425 | 437 | 476 |
| 36 | 284 | 438 | 425 | 478 |
| 37 | 286 | 451 | 435 | 480 |
| 38 | 283 | 422 | 439 | 484 |
| 39 | 290 | 459 | 440 | 482 |
| 40 | 290 | 413 | 428 | 483 |
| 41 | 283 | 503 | 438 | 487 |
| 42 | 290 | 452 | 436 | 470 |
| 43 | 284 | 457 | 427 | 485 |
| 44 | 285 | 426 | 438 | 481 |
| 45 | 288 | 439 | 452 | 483 |
| 46 | 287 | 456 | 453 | 474 |
| 47 | 282 | 475 | 471 | 486 |
| 48 | 286 | 430 | 464 | 483 |
| 49 | 287 | 467 | 434 | 473 |
| 50 | 284 | 471 | 423 | 485 |
| 51 | 286 | 483 | 436 | 483 |
| 52 | 285 | 439 | 454 | 490 |
| 53 | 284 | 428 | 472 | 476 |
| 54 | 283 | 437 | 452 | 474 |
| 55 | 286 | 456 | 450 | 474 |
| 56 | 290 | 457 | 437 | 478 |
| 57 | 288 | 398 | 433 | 483 |
| 58 | 290 | 437 | 424 | 482 |
| 59 | 284 | 426 | 427 | 488 |
| 60 | 287 | 453 | 438 | 478 |
| 61 | 284 | 487 | 442 | 488 |
| 62 | 282 | 474 | 432 | 490 |
| 63 | 284 | 472 | 419 | 484 |
| 64 | 284 | 420 | 445 | 483 |
| 65 | 289 | 431 | 452 | 476 |
| 66 | 290 | 524 | 450 | 487 |
| 67 | 285 | 435 | 461 | 471 |
| 68 | 287 | 436 | 456 | 474 |
| 69 | 285 | 450 | 437 | 468 |
| 70 | 285 | 436 | 439 | 476 |
| 71 | 287 | 468 | 440 | 472 |
| 72 | 289 | 467 | 431 | 474 |
| 73 | 284 | 424 | 430 | 472 |
| 74 | 289 | 427 | 435 | 478 |
| 75 | 290 | 432 | 453 | 480 |
| 76 | 289 | 459 | 447 | 468 |
| 77 | 287 | 460 | 449 | 474 |
| 78 | 285 | 476 | 432 | 471 |
| 79 | 284 | 479 | 426 | 465 |
| 80 | 283 | 490 | 460 | 462 |
| 81 | 290 | 513 | 463 | 476 |
| 82 | 289 | 457 | 452 | 475 |
| 83 | 289 | 458 | 457 | 473 |
| 84 | 288 | 434 | 453 | 482 |
| 85 | 285 | 547 | 431 | 485 |
| 86 | 287 | 450 | 435 | 473 |
| 87 | 289 | 435 | 447 | 468 |
| 88 | 290 | 451 | 446 | 470 |
| 89 | 290 | 456 | 449 | 472 |
| 90 | 293 | 452 | 453 | 484 |
| 91 | 287 | 432 | 437 | 489 |
| 92 | 285 | 424 | 433 | 480 |
| 93 | 284 | 426 | 456 | 475 |
| 94 | 287 | 428 | 451 | 478 |
| 95 | 284 | 431 | 432 | 490 |
| 96 | 286 | 435 | 413 | 485 |
| 97 | 285 | 451 | 425 | 476 |
| 98 | 284 | 402 | 451 | 462 |
| 99 | 290 | 441 | 439 | 478 |
| 100 | 284 | 473 | 457 | 471 |
| Average | 287.31 | 451.38 | 437.49 | 475.35 |
| height | | | | |

As can be seen in Table 5, even with the use of a growth regulator, the Ptilotus of the present invention is unexpectedly shorter and more compact than the three Ptilotus populations. The Ptilotus of the present invention has an average plant height of 287.31 mm, while the Ptilotus populations have a plant height between 451.38 mm and 475.35 mm.

### Example 5

### Days to Flower of the Present Invention without a Growth Regulator

In an example of the present invention, the cultivar of the present invention was grown without a growth regulator and compared with three Ptilotus population. The plants were first grown in plug trays in an unheated poly house. The plants were then transplanted to 10 cm pots and placed outside of the poly house.

Table 6 shows the total days to the first flowering of the present invention when compared with three Ptilotus populations from Australia. Table 6 shows the replication number (column one), the total days to first flowering of the Ptilotus of the present invention (column two), the total days to first flowering of Ptilotus population A (column three), the total days to first flowering of Ptilotus population B (column four) and the total days to first flowering of Ptilotus population C (column five). Sowing of seed was conducted in September 2008 in Macquarie Fields, Australia. The plants were transplanted to 10 cm pots at six weeks and placed outside of the poly house.

TABLE 6
Number of days to first flowering without a growth regulator

| | | Days to 1st | Days to 1st | Days to 1st |
|---|---|---|---|---|
| | Days to 1st | flowering - | flowering - | flowering - |
| | flowering - | Ptilotus | Ptilotus | Ptilotus |
| Plant No. | BEPT107 | population A | population B | population C |
| 1 | 80 | 89 | 106 | 131 |
| 2 | 82 | 93 | 107 | 133 |
| 3 | 82 | 101 | 107 | 134 |
| 4 | 82 | 103 | 109 | 135 |
| 5 | 82 | 103 | 109 | 135 |
| 6 | 82 | 105 | 109 | 137 |
| 7 | 82 | 105 | 110 | 137 |
| 8 | 82 | 107 | 113 | 138 |
| 9 | 84 | 107 | 113 | 138 |
| 10 | 84 | 107 | 115 | 139 |
| 11 | 84 | 107 | 117 | 139 |
| 12 | 84 | 109 | 117 | 139 |
| 13 | 84 | 109 | 119 | 139 |
| 14 | 84 | 109 | 121 | 139 |
| 15 | 84 | 109 | 121 | 139 |
| 16 | 84 | 109 | 121 | 139 |
| 17 | 85 | 109 | 121 | 139 |
| 18 | 85 | 110 | 121 | 139 |
| 19 | 85 | 110 | 121 | 139 |
| 20 | 85 | 110 | 123 | 139 |
| 21 | 85 | 110 | 123 | 139 |
| 22 | 85 | 110 | 123 | 139 |
| 23 | 85 | 111 | 123 | 140 |
| 24 | 85 | 111 | 123 | 140 |
| 25 | 86 | 111 | 125 | 140 |
| 26 | 86 | 111 | 125 | 140 |
| 27 | 86 | 111 | 126 | 140 |
| 28 | 86 | 111 | 126 | 140 |
| 29 | 86 | 111 | 127 | 140 |
| 30 | 86 | 112 | 128 | 140 |
| 31 | 86 | 112 | 133 | 140 |
| 32 | 86 | 112 | 134 | 140 |
| 33 | 86 | 112 | 134 | 140 |
| 34 | 86 | 112 | 134 | 140 |
| 35 | 86 | 113 | 134 | 140 |
| 36 | 86 | 113 | 134 | 140 |
| 37 | 86 | 114 | 134 | 140 |
| 38 | 86 | 115 | 134 | 140 |
| 39 | 86 | 115 | 137 | 140 |
| 40 | 86 | 115 | 137 | 142 |
| 41 | 86 | 116 | 137 | 142 |
| 42 | 86 | 116 | 137 | 142 |
| 43 | 86 | 117 | 138 | 142 |
| 44 | 87 | 117 | 138 | 142 |
| 45 | 87 | 119 | 138 | 142 |
| 46 | 87 | 119 | 138 | 142 |
| 47 | 87 | 119 | 138 | 142 |
| 48 | 87 | 119 | 139 | 142 |
| 49 | 87 | 119 | 139 | 142 |
| 50 | 87 | 119 | 138 | 142 |
| 51 | 87 | 120 | 138 | 142 |
| 52 | 87 | 120 | 138 | 142 |
| 53 | 87 | 120 | 139 | 142 |
| 54 | 87 | 120 | 139 | 142 |
| 55 | 87 | 120 | 139 | 142 |
| 56 | 87 | 120 | 139 | 142 |
| 57 | 87 | 120 | 140 | 142 |
| 58 | 87 | 120 | 140 | 142 |
| 59 | 87 | 121 | 140 | 142 |
| 60 | 87 | 121 | 140 | 142 |
| 61 | 87 | 121 | 140 | 142 |
| 62 | 87 | 121 | 141 | 142 |
| 63 | 87 | 121 | 142 | 142 |
| 64 | 87 | 122 | 142 | 142 |
| 65 | 87 | 122 | 142 | 142 |
| 66 | 87 | 122 | 143 | 143 |
| 67 | 87 | 122 | 143 | 143 |
| 68 | 87 | 124 | 143 | 143 |
| 69 | 87 | 124 | 143 | 144 |
| 70 | 87 | 124 | 143 | 144 |
| 71 | 87 | 124 | 143 | 144 |
| 72 | 87 | 124 | 143 | 144 |
| 73 | 87 | 124 | 144 | 144 |
| 74 | 87 | 124 | 144 | 146 |
| 75 | 87 | 124 | 144 | 146 |
| 76 | 87 | 125 | 144 | 146 |
| 77 | 87 | 125 | 144 | 146 |
| 78 | 87 | 125 | 144 | 146 |
| 79 | 87 | 125 | 144 | 146 |
| 80 | 87 | 125 | 144 | 146 |
| 81 | 88 | 127 | 144 | 146 |
| 82 | 88 | 127 | 144 | 146 |
| 83 | 88 | 128 | 144 | 146 |
| 84 | 88 | 128 | 144 | 146 |
| 85 | 88 | 128 | 145 | 148 |
| 86 | 89 | 130 | 145 | 148 |
| 87 | 89 | 131 | 145 | 148 |
| 88 | 89 | 135 | 145 | 148 |
| 89 | 89 | 137 | 146 | 148 |
| 90 | 89 | 137 | 146 | 148 |
| 91 | 89 | 145 | 146 | 148 |
| 92 | 89 | 145 | 146 | 148 |
| 93 | 89 | 147 | 147 | 148 |
| 94 | 89 | 149 | 147 | 148 |
| 95 | 89 | 156 | 147 | 148 |
| 96 | 89 | 157 | 147 | 148 |
| 97 | 90 | 157 | 147 | 148 |
| 98 | 90 | 160 | 147 | 148 |
| 99 | 93 | 161 | 149 | 148 |
| 100 | 95 | 172 | 150 | 151 |
| Average | 86.46 | 120.6 | 134.47 | 142.23 |
| number of | | | | |
| days to flower | | | | |

As can be seen in Table 6, the Ptilotus of the present invention has an unexpectedly shorter number of days to flower than the three comparison Ptilotus populations. The number of days to flower of the present invention averaged 86.46, while the three Ptilotus populations averaged between 120.6 days and 142.23 days.

### Example 6

### Days to First Flower of the Present Invention with a Growth Regulator

In an example of the present invention, the cultivar of the present invention was grown without a growth regulator and compared with three Ptilotus populations. The plants were first grown in plug trays in an unheated poly house. The plants were then transplanted to 10 cm pots and placed outside of the poly house. The growth regulator Regalis, with an active substance of Prohexadion-Ca, was applied twice to the plants at a concentration of 0.5 g/litre. The Regalis was applied as a drench on the plug trays at five weeks, which was one week prior to potting of the plants. The second application was applied two weeks after potting.

Table 7 shows the total days to the first flowering of the present invention when compared with three Ptilotus populations from Australia. Table 7 shows the replication number (column one), the total days to first flowering of the Ptilotus of the present invention (column two), the total days to first flowering of Ptilotus population A (column three), the total days to first flowering of Ptilotus population B (column four) and the total days to first flowering of Ptilotus population C (column five). Sowing of seed was conducted September 2008 in Macquarie Fields, Australia. The plants were transplanted to 10 cm pots at six weeks and placed outside of the poly house.

TABLE 7
Number of days to first flowering using a growth regulator

| | Days | | | |
|---|---|---|---|---|
| | to first | Days to first | Days to first | Days to first |
| | flowering | flowering with | flowering with | flowering with |
| | with | growth | growth | growth |
| | growth | regulator - | regulator - | regulator - |
| | regulator - | Ptilotus | Ptilotus | Ptilotus |
| Plant No. | BEPT107 | population A | population B | population C |
| 1 | 88 | 101 | 110 | 140 |
| 2 | 88 | 111 | 117 | 142 |
| 3 | 90 | 111 | 121 | 142 |
| 4 | 90 | 112 | 121 | 144 |
| 5 | 90 | 115 | 121 | 144 |
| 6 | 90 | 120 | 122 | 144 |
| 7 | 93 | 120 | 122 | 144 |
| 8 | 93 | 121 | 122 | 144 |
| 9 | 93 | 121 | 122 | 145 |
| 10 | 93 | 123 | 124 | 146 |
| 11 | 93 | 123 | 124 | 146 |
| 12 | 93 | 124 | 124 | 146 |
| 13 | 93 | 125 | 126 | 147 |
| 14 | 93 | 126 | 128 | 147 |
| 15 | 93 | 126 | 130 | 147 |
| 16 | 93 | 126 | 134 | 147 |
| 17 | 93 | 126 | 134 | 147 |
| 18 | 93 | 127 | 135 | 147 |
| 19 | 93 | 127 | 135 | 148 |
| 20 | 93 | 127 | 138 | 148 |
| 21 | 93 | 127 | 138 | 148 |
| 22 | 93 | 127 | 138 | 148 |
| 23 | 93 | 127 | 138 | 148 |
| 24 | 93 | 127 | 138 | 148 |
| 25 | 93 | 127 | 138 | 148 |
| 26 | 95 | 127 | 138 | 148 |
| 27 | 95 | 127 | 138 | 148 |
| 28 | 95 | 128 | 138 | 148 |
| 29 | 95 | 128 | 138 | 149 |
| 30 | 95 | 128 | 141 | 149 |
| 31 | 95 | 128 | 141 | 149 |
| 32 | 95 | 128 | 141 | 149 |
| 33 | 95 | 128 | 141 | 149 |
| 34 | 95 | 128 | 144 | 149 |
| 35 | 95 | 128 | 144 | 149 |
| 36 | 95 | 129 | 144 | 149 |
| 37 | 95 | 129 | 144 | 149 |
| 38 | 95 | 129 | 146 | 149 |
| 39 | 95 | 130 | 148 | 149 |
| 40 | 95 | 131 | 150 | 149 |
| 41 | 95 | 131 | 150 | 149 |
| 42 | 95 | 131 | 150 | 149 |
| 43 | 95 | 133 | 150 | 149 |
| 44 | 95 | 133 | 150 | 149 |
| 45 | 95 | 133 | 150 | 149 |
| 46 | 95 | 133 | 150 | 149 |
| 47 | 95 | 133 | 150 | 149 |
| 48 | 95 | 133 | 150 | 149 |
| 49 | 95 | 133 | 152 | 149 |
| 50 | 95 | 135 | 152 | 149 |
| 51 | 95 | 135 | 152 | 149 |
| 52 | 95 | 135 | 152 | 149 |
| 53 | 95 | 135 | 152 | 149 |
| 54 | 95 | 136 | 152 | 150 |
| 55 | 95 | 136 | 152 | 150 |
| 56 | 95 | 136 | 152 | 150 |
| 57 | 95 | 137 | 152 | 150 |
| 58 | 95 | 137 | 153 | 150 |
| 59 | 95 | 137 | 153 | 150 |
| 60 | 95 | 137 | 153 | 150 |
| 61 | 95 | 137 | 153 | 150 |
| 62 | 95 | 137 | 153 | 150 |
| 63 | 95 | 137 | 153 | 150 |
| 64 | 95 | 138 | 153 | 151 |
| 65 | 95 | 138 | 153 | 152 |
| 66 | 95 | 138 | 153 | 152 |
| 67 | 95 | 138 | 153 | 152 |
| 68 | 95 | 138 | 153 | 152 |
| 69 | 95 | 138 | 154 | 152 |
| 70 | 95 | 138 | 154 | 152 |
| 71 | 95 | 139 | 154 | 152 |
| 72 | 95 | 139 | 154 | 152 |
| 73 | 95 | 139 | 154 | 152 |
| 74 | 95 | 139 | 154 | 152 |
| 75 | 95 | 139 | 154 | 152 |
| 76 | 95 | 139 | 154 | 152 |
| 77 | 95 | 139 | 154 | 152 |
| 78 | 95 | 139 | 154 | 152 |
| 79 | 95 | 139 | 154 | 153 |
| 80 | 95 | 139 | 154 | 154 |
| 81 | 95 | 139 | 154 | 154 |
| 82 | 95 | 139 | 154 | 154 |
| 83 | 95 | 141 | 154 | 154 |
| 84 | 95 | 142 | 154 | 154 |
| 85 | 95 | 143 | 154 | 157 |
| 86 | 95 | 143 | 156 | 157 |
| 87 | 95 | 143 | 156 | 157 |
| 88 | 95 | 143 | 156 | 157 |
| 89 | 95 | 143 | 156 | 157 |
| 90 | 95 | 144 | 158 | 157 |
| 91 | 95 | 144 | 158 | 157 |
| 92 | 97 | 144 | 158 | 157 |
| 93 | 97 | 144 | 158 | 158 |
| 94 | 97 | 144 | 160 | 159 |
| 95 | 97 | 144 | 160 | 159 |
| 96 | 97 | 146 | 160 | 159 |
| 97 | 97 | 147 | 160 | 161 |
| 98 | 100 | 148 | 160 | 162 |
| 99 | 102 | 150 | 160 | 165 |
| 100 | 102 | 151 | 162 | 167 |
| Average | 94.59 | 133.11 | 145.83 | 150.62 |
| number | | | | |
| of days | | | | |
| to flower | | | | |

As can be seen in Table 7, the Ptilotus of the present invention has an unexpectedly shorter number of days to flower than the three comparison Ptilotus populations when grown with a growth regulator. The number of days to flower of the present invention averaged 94.59 days, while the three Ptilotus populations averaged between 133.11 days and 150.62 days.

This invention is also directed to methods for producing a Ptilotus plant by crossing a first parent Ptilotus plant with a second parent Ptilotus plant, wherein the first or second Ptilotus plant is the Ptilotus plant from the present invention. Further, both first and second parent Ptilotus plants may be from the present invention. Therefore, any methods using the present invention are part of this invention: selfing, backcrosses, hybrid breeding, and crosses to populations. Any plants produced using the present invention as at least one parent is within the scope of this invention.

Additional methods include, but are not limited to, expression vectors introduced into plant tissues using a direct gene transfer method such as microprojectile-mediated delivery, DNA injection, electroporation and the like. More preferably, expression vectors are introduced into plant tissues by using either microprojectile-mediated delivery with a biolistic device or by using Agrobacterium-mediated transformation. Transformant plants obtained with the protoplasm of the invention are intended to be within the scope of this invention.

### Further Embodiments of the Invention

The advent of new molecular biological techniques has allowed the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner. Any DNA sequences, whether from a different species or from the same species, which are inserted into the genome using transformation, are referred to herein collectively as “transgenes.” In some embodiments of the invention, a transgenic variant of the present invention may contain at least one transgene but could contain at least 1, 2, 3, 4, 5, 6, 7, 8, 9, 10 and/or no more than 15, 14, 13, 12, 11, 10, 9, 8, 7, 6, 5, 4, 3, or 2. Over the last fifteen to twenty years several methods for producing transgenic plants have been developed, and the present invention also relates to transgenic variants of the claimed improved Ptilotus plant.

One embodiment of the invention is a process for producing the present invention further comprising a desired trait, said process comprising transforming the present invention with a transgene that confers a desired trait. Another embodiment is the product produced by this process. In one embodiment, the desired trait may be one or more of decreased days to flower, compact habit, increased germination rate, herbicide resistance, insect resistance, or disease resistance. The specific gene may be any known in the art or listed herein, including: a polynucleotide conferring resistance to imidazolinone, sulfonylurea, glyphosate, glufosinate, triazine, benzonitrile, cyclohexanedione, phenoxy proprionic acid and L-phosphinothricin; a polynucleotide encoding a Bacillus thuringiensis polypeptide, a polynucleotide encoding phytase, FAD-2, FAD-3, galactinol synthase or a raffinose synthetic enzyme; or a polynucleotide conferring resistance to Phytophthora root rot.

Numerous methods for plant transformation have been developed, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pp. 67-88 and Armstrong, “The First Decade of Maize Transformation: A Review and Future Perspective” (Maydica 44:101-109, 1999). In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

A genetic trait which has been engineered into the genome of a particular Ptilotus plant may then be moved into the genome of another variety using traditional breeding techniques that are well known in the plant breeding arts. For example, a backcrossing approach is commonly used to move a transgene from a transformed Ptilotus variety into an already developed Ptilotus variety, and the resulting backcross conversion plant would then comprise the transgene(s).

Various genetic elements can be introduced into the plant genome using transformation. These elements include, but are not limited to, genes, coding sequences, inducible, constitutive, and tissue specific promoters, enhancing sequences, and signal and targeting sequences. For example, see the traits, genes and transformation methods listed in U.S. Pat. No. 6,118,055.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed Ptilotus plants using transformation methods as described below to incorporate transgenes into the genetic material of the Ptilotus plant(s).

### Expression Vectors for Ptilotus Transformation: Marker Genes

Expression vectors include at least one genetic marker operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. USA, 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant (Hayford et al., Plant Physiol. 86:1216 (1988), Jones et al., Mol. Gen. Genet., 210:86 (1987), Svab et al., Plant Mol. Biol. 14:197 (1990), Hille et al., Plant Mol. Biol. 7:171 (1986)). Other selectable marker genes confer resistance to herbicides such as glyphosate, glufosinate or bromoxynil (Comai et al., Nature 317:741-744 (1985), Gordon-Kamm et al., Plant Cell 2:603-618 (1990) and Stalker et al., Science 242:419-423 (1988)).

Selectable marker genes for plant transformation not of bacterial origin include, for example, mouse dihydrofolate reductase, plant 5-enolpyruvylshikimate-3-phosphate synthase and plant acetolactate synthase (Eichholtz et al., Somatic Cell Mol. Genet. 13:67 (1987), Shah et al., Science 233:478 (1986), Charest et al., Plant Cell Rep. 8:643 (1990)).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS), β-galactosidase, luciferase and chloramphenicol acetyltransferase (Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984)).

In vivo methods for visualizing GUS activity that do not require destruction of plant tissue are available (Molecular Probes publication 2908, IMAGENE GREEN, pp. 1-4 (1993) and Naleway et al., J. Cell Biol. 115:151a (1991)). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells (Chalfie et al., Science 263:802 (1994)). GFP and mutants of GFP may be used as screenable markers.

### Expression Vectors for Ptilotus Transformation: Promoters

Genes included in expression vectors must be driven by a nucleotide sequence comprising a regulatory element, (for example, a promoter). Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in a certain tissue are referred to as “tissue-specific.” A “cell-type” specific promoter primarily drives expression in certain cell types in one or more organs, for example, vascular cells in roots or leaves. An “inducible” promoter is a promoter which is under environmental control. Examples of environmental conditions that may effect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions.

A. Inducible Promoters: An inducible promoter is operably linked to a gene for expression in Ptilotus. Optionally, the inducible promoter is operably linked to a nucleotide sequence encoding a signal sequence which is operably linked to a gene for expression in Ptilotus. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Mett et al., Proc. Natl. Acad. Sci. USA 90:4567-4571 (1993)); In2 gene from maize which responds to benzenesulfonamide herbicide safeners (Hershey et al., Mol. Gen. Genetics 227:229-237 (1991) and Gatz et al., Mol. Gen. Genetics 243: 32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. USA 88:0421 (1991)).

B. Constitutive Promoters: A constitutive promoter is operably linked to a gene for expression in Ptilotus or the constitutive promoter is operably linked to a nucleotide sequence encoding a signal sequence, which is operably linked to a gene for expression in Ptilotus.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3):291-300 (1992)). The ALS promoter, Xba1/Ncol fragment 5′ to the Brassica napus ALS3 structural gene (or a nucleotide sequence similarity to said Xba1/Ncol fragment), represents a particularly useful constitutive promoter. See PCT Application WO 96/30530.

C. Tissue-specific or Tissue-preferred Promoter: A tissue-specific promoter is operably linked to a gene for expression in Ptilotus. Optionally, the tissue-specific promoter is operably linked to a nucleotide sequence encoding a signal sequence which is operably linked to a gene for expression in Ptilotus. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of a protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine during protein synthesis and processing where the encoded protein is ultimately compartmentalized.

The presence of a signal sequence directs a polypeptide to either an intracellular organelle or subcellular compartment or for secretion to the apoplast. Many signal sequences are known in the art. See, for example, Becker et al., Plant Mol. Biol. 20:49 (1992); Knox, C., et al., Plant Mol. Biol. 9:3-17 (1987); Lerner et al., Plant Physiol. 91:124-129 (1989); Frontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Foreign Protein Genes and Horticultural Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to a preferred embodiment, the transgenic plant provided for commercial production of foreign protein is a Ptilotus plant. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology, CRC Press, Boca Raton 269:284 (1993). Map information concerning chromosomal location is useful for proprietary protection of a subject transgenic plant.

Wang et al. discuss “Large Scale Identification, Mapping and Genotyping of Single-Nucleotide Polymorphisms in the Human Genome,” Science, 280:1077-1082, 1998, and similar capabilities are becoming increasingly available for the Ptilotus genome. Map information concerning chromosomal location is useful for proprietary protection of a subject transgenic plant. If unauthorized propagation is undertaken and crosses made with other germplasm, the map of the integration region can be compared to similar maps for suspect plants to determine if the latter have a common parentage with the subject plant. Map comparisons would involve hybridizations, RFLP, PCR, SSR and sequencing, all of which are conventional techniques. SNPs may also be used alone or in combination with other techniques.

Likewise, by means of the present invention, plants can be genetically engineered to express various phenotypes of horticultural interest. Through the transformation of Ptilotus the expression of genes can be altered to enhance disease resistance, insect resistance, herbicide resistance, and other traits. DNA sequences native to Ptilotus as well as non-native DNA sequences can be transformed into Ptilotus and used to alter levels of native or non-native proteins. Various promoters, targeting sequences, enhancing sequences, and other DNA sequences can be inserted into the genome for the purpose of altering the expression of proteins. Reduction of the activity of specific genes (also known as gene silencing, or gene suppression) is desirable for several aspects of genetic engineering in plants.

Many techniques for gene silencing are well known to one of skill in the art, including but not limited to knock-outs (such as by insertion of a transposable element such as mu (Vicki Chandler, The Maize Handbook, Ch. 118 (Springer-Verlag 1994) or other genetic elements such as a FRT, Lox or other site specific integration site, antisense technology (see, e.g., Sheehy et al. (1988) PNAS USA 85:8805-8809; and U.S. Pat. Nos. 5,107,065; 5,453,566; and 5,759,829); co-suppression (e.g., Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) PNAS USA 95:15502-15507), virus-induced gene silencing (Burton et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman & Sakai (2003) Plant Cell 15:2730-2741); ribozymes (Steinecke et al. (1992) EMBO J. 11:1525; and Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); and other methods or combinations of the above methods known to those of skill in the art.

Likewise, by means of the present invention, horticultural genes can be expressed in transformed plants. More particularly, plants can be genetically engineered to express various phenotypes of horticultural interest. Exemplary genes implicated in this regard include, but are not limited to, those categorized below:

1. Genes that Confer Resistance to Pests or Disease and that Encode

A. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example, Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al. Cell 78:1089 (1994) (Arabidopsis RSP2 gene for resistance to Pseudomonas syringae), McDowell & Woffenden, (2003) Trends Biotechnol. 21(4): 178-83 and Toyoda et al., (2002) Transgenic Res. 11 (6):567-82.

B. A gene conferring resistance to a pest, such as a nematode. See e.g., PCT Application WO 96/30517; PCT Application WO 93/19181.

C. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, Van Damme et al., Plant Molec. Biol. 24:25 (1994), who disclose the nucleotide sequences of several Clivia miniata mannose-binding lectin genes.

E. A vitamin-binding protein such as avidin. See PCT Application US 93/06487 which teaches the use of avidin and avidin homologues as larvicides against insect pests.

F. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor); Huub et al., Plant Molec. Biol. 21:985 (1993) (nucleotide sequence of cDNA encoding tobacco proteinase inhibitor I) Sumitani et al., Biosci. Biotech. Biochem. 57:1243 (1993) (nucleotide sequence of Streptomyces nitrosporeus α-amylase inhibitor) and U.S. Pat. No. 5,494,813 (Hepher and Atkinson, issued Feb. 27, 1996).

G. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata); Chattopadhyay et al. (2004) Critical Reviews in Microbiology 30 (1): 33-54 2004; Zjawiony (2004) J Nat Prod 67 (2): 300-310; Carlini & Grossi-de-Sa (2002) Toxicon, 40 (11): 1515-1539; Ussuf et al. (2001) Curr Sci. 80 (7): 847-853; and Vasconcelos & Oliveira (2004) Toxicon 44 (4): 385-403. See also U.S. Pat. No. 5,266,317 to Tomalski et al., which discloses genes encoding insect-specific, paralytic neurotoxins.

I. An insect-specific venom produced in nature by a snake, a wasp, etc. For example, see Pang et al., Gene 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT Application WO 93/02197 (Scott et al.), which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene, U.S. Pat. Nos. 7,145,060, 7,087,810 and 6,563,020.

L. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

M. A hydrophobic moment peptide. See PCT Application WO 95/16776 and U.S. Pat. No. 5,580,852, which disclose peptide derivatives of tachyplesin which inhibit fungal plant pathogens, and PCT Application WO 95/18855 and U.S. Pat. No. 5,607,914 which teaches synthetic antimicrobial peptides that confer disease resistance.

N. A membrane permease, a channel former or a channel blocker. For example, see the disclosure of Jaynes et al., Plant Sci 89:43 (1993), of heterologous expression of a cecropin-β lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus and tobacco mosaic virus.

P. An insect-specific antibody or an immunotoxin derived therefrom. Thus, an antibody targeted to a critical metabolic function in the insect gut would inactivate an affected enzyme, killing the insect. See Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

Q. A virus-specific antibody. See, for example, Tavladoraki et al., Nature 366:469 (1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

S. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

T. Genes involved in the Systemic Acquired Resistance (SAR) Response and/or the pathogenesis-related genes. Briggs, S., Current Biology, 5(2) (1995); Pieterse & Van Loon (2004) Curr. Opin. Plant Bio. 7(4):456-64 and Somssich (2003) Cell 113(7):815-6.

U. Antifungal genes. See Cornelissen and Melchers, Plant Physiol., 101:709-712 (1993); Parijs et al., Planta 183:258-264 (1991) and Bushnell et al., Can. J. of Plant Path. 20(2):137-149 (1998). Also see U.S. Pat. No. 6,875,907.

V. Detoxification genes, such as for fumonisin, beauvericin, moniliformin and zearalenone and their structurally related derivatives. For example, see U.S. Pat. No. 5,792,931.

W. Cystatin and cysteine proteinase inhibitors. See U.S. Pat. No. 7,205,453.

X. Defensin genes. See WO 03/000863 and U.S. Pat. No. 6,911,577.

Y. Genes that confer resistance to Phytophthora root rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7 and other Rps genes. See, for example, Shoemaker et al., Phytophthora Root Rot Resistance Gene Mapping in Soybean, Plant Genome IV Conference, San Diego, Calif. (1995).

2. Genes that Confer Resistance to an Herbicide, for Example

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvlshikimate-3-phosphate synthase (EPSPS) and aroA genes, respectively) and other phosphono compounds such as glufosinate (phosphinothricin acetyl transferase (PAT) and Streptomyces hygroscopicus PAT bar genes), and pyridinoxy or phenoxy proprionic acids and cyclohexanediones (ACCase inhibitor-encoding genes). See, for example, U.S. Pat. No. 4,940,835 to Shah et al., which discloses the nucleotide sequence of a form of EPSPS which can confer glyphosate resistance. U.S. Pat. No. 5,627,061 to Barry et al. also describes genes encoding EPSPS enzymes. See also U.S. Pat. Nos. 6,566,587; 6,338,961; 6,248,876 B1; 6,040,497; 5,804,425; 5,633,435; 5,145,783; 4,971,908; 5,312,910; 5,188,642; 4,940,835; 5,866,775; 6,225,114 B1; 6,130,366; 5,310,667; 4,535,060; 4,769,061; 5,633,448; 5,510,471; Re. 36,449; RE 37,287 E; and 5,491,288; and international publications EP1173580; WO 01/66704; EP1173581 and EPI 173582, which are incorporated herein by reference for this purpose. Glyphosate resistance is also imparted to plants that express a gene that encodes a glyphosate oxido-reductase enzyme as described more fully in U.S. Pat. Nos. 5,776,760 and 5,463,175, which are incorporated herein by reference for this purpose. In addition glyphosate resistance can be imparted to plants by the over expression of genes encoding glyphosate N-acetyltransferase. See, for example, U.S. application Ser. No. 10/427,692. A DNA molecule encoding a mutant aroA gene can be obtained under ATCC Accession No. 39256, and the nucleotide sequence of the mutant gene is disclosed in U.S. Pat. No. 4,769,061 to Comai. European Patent Application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European Application No. 0 242 246 to Leemans et al. DeGreef et al., Bio/Technology 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall et al., Theor. Appl. Genet. 83:435 (1992).

C. An herbicide that inhibits photosynthesis, such as a triazine (psbA and gs+ genes) and a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Nos. 53435, 67441 and 67442. Cloning and expression of DNA coding for a glutathione S-transferase is described by Hayes et al., Biochem. J. 285:173 (1992).

D. Acetohydroxy acid synthase, which has been found to make plants that express this enzyme resistant to multiple types of herbicides, has been introduced into a variety of plants. See Hattori et al., Mol. Gen. Genet. 246:419, (1995). Other genes that confer tolerance to herbicides include a gene encoding a chimeric protein of rat cytochrome P4507A1 and yeast NADPH-cytochrome P450 oxidoreductase (Shiota et al., Plant Physiol., 106:17, 1994), genes for glutathione reductase and superoxide dismutase (Aono et al., Plant Cell Physiol. 36:1687, 1995), and genes for various phosphotransferases (Datta et al., Plant Mol. Biol. 20:619, 1992).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306; 6,282,837; 5,767,373; and international publication WO 01/12825.

3. Genes that Create a Site for Site Specific DNA Integration.

This includes the introduction of FRT sites that may be used in the FLP/FRT system and/or Lox sites that may be used in the Cre/Loxp system. For example, see Lyznik, et al., Site-Specific Recombination for Genetic Engineering in Plants, Plant Cell Rep (2003) 21:925-932 and WO 99/25821, which are hereby incorporated by reference. Other systems that may be used include the Gin recombinase of phage Mu (Maeser et al., 1991; Vicki Chandler, The Maize Handbook Ch. 118 (Springer-Verlag 1994), the Pin recombinase of E. coli (Enomoto et al., 1983), and the R/RS system of the pSR1 plasmid (Araki et al., 1992).

4. Genes that Affect Abiotic Stress Resistance.

Genes that affect abiotic stress resistance (including, but not limited, to flowering, seed development, drought resistance or tolerance, cold resistance or tolerance, and salt resistance or tolerance) and increased yield under stress. For example, see: WO 00/73475 where water use efficiency is altered through alteration of malate; U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 98/09521, and WO 99/38977 describing genes, including CBF genes and transcription factors effective in mitigating the negative effects of freezing, high salinity, and drought on plants, as well as conferring other positive effects on plant phenotype; US 2004/0148654 and WO 01/36596 where abscisic acid is altered in plants resulting in improved plant phenotype such as increased tolerance to abiotic stress; WO 2000/006341, WO 04/090143, U.S. application Ser. No. 10/817,483 and U.S. Pat. No. 6,992,237 where cytokinin expression is modified resulting in plants with increased stress tolerance, such as drought tolerance. For ethylene alteration, see US 20040128719, US 20030166197 and WO 2000/32761. For plant transcription factors or transcriptional regulators of abiotic stress, see, e.g., US 20040098764 or US 20040078852.

Other genes and transcription factors that affect plant growth and horticultural traits such as flowering, plant growth and/or plant structure, can be introduced or introgressed into plants, see, e.g., WO 97/49811 (LHY), WO 98/56918 (ESD4), WO 97/10339, U.S. Pat. No. 6,573,430 (TFL), U.S. Pat. No. 6,713,663 (FT), WO 96/14414 (CON), WO 96/38560, WO 01/21822 (VRN1), WO 00/44918 (VRN2), WO 99/49064 (GI), WO 00/46358 (FRI), WO 97/29123, U.S. Pat. No. 6,794,560, U.S. Pat. No. 6,307,126 (GAI), WO 99/09174 (D8 and Rht), WO 2004/076638, and WO 2004/031349 (transcription factors).

### Methods for Ptilotus Transformation

Numerous methods for plant transformation have been developed including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pp. 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pp. 89-119.

A. Agrobacterium-mediated Transformation—One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996.

B. Direct Gene Transfer—Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987); Sanford, J. C., Trends Biotech. 6:299 (1988); Klein et al., Bio/Tech. 6:559-563 (1988); Sanford, J. C. Physiol Plant 7:206 (1990); Klein et al., Biotechnology 10:268 (1992). See also U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang et al., Bio/Technology 9:996 (1991). Alternatively, liposome and spheroplast fusion have been used to introduce expression vectors into plants. Deshayes et al., EMBO J., 4:2731 (1985); Christou et al., Proc Natl. Acad. Sci. USA 84:3962 (1987). Direct uptake of DNA into protoplasts using CaCl₂ precipitation, polyvinyl alcohol or poly-L-ornithine have also been reported. Hain et al., Mol. Gen. Genet. 199:161 (1985) and Draper et al., Plant Cell Physiol. 23:451 (1982). Electroporation of protoplasts and whole cells and tissues have also been described (Donn et al., In Abstracts of VIIth International Congress on Plant Cell and Tissue Culture IAPTC, A2-38, pp. 53 (1990); D'Halluin et al., Plant Cell 4:1495-1505 (1992) and Spencer et al., Plant Mol. Biol. 24:51-61 (1994)).

Following transformation of Ptilotus target tissues, expression of the above-described selectable marker genes allows for preferential selection of transformed cells, tissues and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular Ptilotus line using the foregoing transformation techniques could be moved into another line using traditional backcrossing techniques that are well known in the plant breeding arts. For example, a backcrossing approach could be used to move an engineered trait from a wild variety into a commercial variety, or from a variety containing a foreign gene in its genome into a variety or varieties that do not contain that gene. As used herein, “crossing” can refer to a simple X by Y cross or the process of backcrossing depending on the context.

### Genetic Marker Profile Through SSR and First Generation Progeny

In addition to phenotypic observations, a plant can also be identified by its genotype. The genotype of a plant can be characterized through a genetic marker profile which can identify plants of the same variety or a related variety or be used to determine or validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as Microsatellites, and Single Nucleotide Polymorphisms (SNPs). For example, see Lee et al. 2007, Molecular taxonomic clarification of Ptilotus exaltatus and Ptilotus nobilis (Amaranthaceae), Australian Systematic Botany, 20:72-81 which is incorporated by reference herein in its entirety.

Particular markers used for these purposes are not limited to any particular set of markers, but are envisioned to include any type of marker and marker profile which provides a means of distinguishing varieties. One method of comparison is to use only homozygous loci for the present invention.

The present invention comprises a Ptilotus plant characterized by molecular and physiological data obtained from the representative sample of said variety deposited with NCIMB. Further provided by the invention is a Ptilotus plant formed by the combination of the disclosed Ptilotus plant or plant cell with another Ptilotus plant or cell and comprising the homozygous alleles of the variety.

Means of performing genetic marker profiles using SSR polymorphisms are well known in the art. SSRs are genetic markers based on polymorphisms in repeated nucleotide sequences, such as microsatellites. A marker system based on SSRs can be highly informative in linkage analysis relative to other marker systems in that multiple alleles may be present. Another advantage of this type of marker is that, through use of flanking primers, detection of SSRs can be achieved, for example, by the polymerase chain reaction (PCR), thereby eliminating the need for labor-intensive Southern hybridization. The PCR detection is done by use of two oligonucleotide primers flanking the polymorphic segment of repetitive DNA. Repeated cycles of heat denaturation of the DNA followed by annealing of the primers to their complementary sequences at low temperatures, and extension of the annealed primers with DNA polymerase, comprise the major part of the methodology.

Following amplification, markers can be scored by electrophoresis of the amplification products. Scoring of marker genotype is based on the size of the amplified fragment, which may be measured by the number of base pairs of the fragment. While variation in the primer used or in laboratory procedures can affect the reported fragment size, relative values should remain constant regardless of the specific primer or laboratory used. When comparing varieties it is preferable if all SSR profiles are performed in the same lab.

The SSR profile of the present invention can be used to identify plants comprising the present invention as a parent, since such plants will comprise the same homozygous alleles as the present invention. Because the present invention is essentially homozygous at all relevant loci, most loci should have only one type of allele present. In contrast, a genetic marker profile of an F₁ progeny should be the sum of those parents, e.g., if one parent was homozygous for allele x at a particular locus, and the other parent homozygous for allele y at that locus, then the F₁ progeny will be xy (heterozygous) at that locus. Subsequent generations of progeny produced by selection and breeding are expected to be of genotype x (homozygous), y (homozygous), or xy (heterozygous) for that locus position. When the F₁ plant is selfed or sibbed for successive filial generations, the locus should be either x or y for that position.

In addition, plants and plant parts substantially benefiting from the use of the present invention in their development, such as the present invention comprising a backcross conversion, transgene, or genetic sterility factor, may be identified by having a molecular marker profile with a high percent identity to the present invention. Such a percent identity might be 95%, 96%, 97%, 98%, 99%, 99.5% or 99.9% identical to the present invention.

The SSR profile of the present invention also can be used to identify essentially derived varieties and other progeny varieties developed from the use of the present invention, as well as cells and other plant parts thereof. Progeny plants and plant parts produced using the present invention may be identified by having a molecular marker profile of at least 25%, 30%, 35%, 40%, 45%, 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 99.5% genetic contribution from the present invention, as measured by either percent identity or percent similarity. Such progeny may be further characterized as being within a pedigree distance of the present invention, such as within 1, 2, 3, 4 or 5 or less cross-pollinations to a Ptilotus plant other than the present invention or a plant that has the present invention as a progenitor. Unique molecular profiles may be identified with other molecular tools such as SNPs and RFLPs.

While determining the SSR genetic marker profile of the plants described supra, several unique SSR profiles may also be identified which did not appear in either parent of such plant. Such unique SSR profiles may arise during the breeding process from recombination or mutation. A combination of several unique alleles provides a means of identifying a plant variety, an F₁ progeny produced from such variety, and progeny produced from such variety.

Introduction of a New Trait or Locus into the Present Invention

The present invention represents a new base genetic variety into which a new locus or trait may be introgressed. Direct transformation and backcrossing represent two important methods that can be used to accomplish such an introgression. The term backcross conversion and single locus conversion are used interchangeably to designate the product of a backcrossing program.

### Tissue Culture

Further reproduction of the variety can occur by tissue culture and regeneration. For example, reference may be had to Williams, R. R., et al. (1989), Cultivation of the pink mulla mulla Ptilotus exaltatus Nees. 1. Seed germination and dormancy, Scientia Hort., 40:267-274; Hennig, F., et al. (1993). Untersuchungen zur Gewebevermehrung (von P. exaltatus und P. obovatus), Dt. Gartenbau 47:2325-2326; and Hentig, W. U., et al. (1995), The development of Ptilotus exaltatus R. Br. under central European conditions, Acta Hort. 397:163-180. Thus, another aspect of this invention is to provide cells which upon growth and differentiation produce Ptilotus plants having the physiological and morphological characteristics of the present invention.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, petioles, leaves, stems, roots, root tips, anthers, pistils and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

### Using the Present Invention to Develop Other Ptilotus Varieties

The present invention also provides a source of breeding material that may be used to develop new Ptilotus varieties. Plant breeding techniques known in the art and used in a Ptilotus plant breeding program include, but are not limited to, recurrent selection, mass selection, bulk selection, backcrossing, pedigree breeding, open pollination breeding, restriction fragment length polymorphism enhanced selection, genetic marker enhanced selection, and transformation. Often combinations of these techniques are used. There are many analytical methods available to evaluate a new variety. The oldest and most traditional method of analysis is the observation of phenotypic traits but genotypic analysis may also be used.

One of ordinary skill in the art of plant breeding would know how to evaluate the traits of two plant varieties to determine if there is no significant difference between the two traits expressed by those varieties. For example, see Fehr and Walt, Principles of Cultivar Development, pp. 261-286 (1987). Thus the invention includes the present invention progeny Ptilotus plants comprising a combination of at least two traits of the present invention selected from the group consisting of those listed in Tables 1 and 2 or the present invention combination of traits listed in the Summary of the Invention, so that said progeny Ptilotus plant is not significantly different for said traits than the present invention as determined at the 5% significance level when grown in the same environmental conditions. Using techniques described herein, molecular markers may be used to identify said progeny plant as a progeny plant of the present invention. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of the present invention may also be characterized through their filial relationship with the present invention as for example, being within a certain number of breeding crosses of the present invention. A breeding cross is a cross made to introduce new genetics into the progeny, and is distinguished from a cross, such as a self or a sib cross, made to select among existing genetic alleles. The lower the number of breeding crosses in the pedigree, the closer the relationship between the present invention and its progeny. For example, progeny produced by the methods described herein may be within 1, 2, 3, 4 or 5 breeding crosses of the present invention.

As used herein, the term “plant” includes plant cells, plant protoplasts, plant cell tissue cultures from which Ptilotus plants can be regenerated, plant calli, plant clumps, and plant cells that are intact in plants or parts of plants, such as embryos, pollen, ovules, flowers, leaves, roots, root tips, anthers, cotyledons, hypocotyls, meristematic cells, stems, pistils, petiole, and the like.

### Deposit Information

A deposit of the Ernst Benary Samenzucht GmbH Ptilotus variety named BEPT107 disclosed above and recited in the appended claims has been made with the National Collections of Industrial, Food and Marine Bacteria (NCIMB), NCIMB Ltd., Ferguson Building, Craibstone Estate, Bucksburn, Aberdeen AB21 9YA, Scotland, UK. The date of deposit was Nov. 26, 2007. The deposit of 2,500 seeds was taken from the same deposit maintained by Ernst Benary Samenzucht GmbH since prior to the filing date of this application. All restrictions upon the deposit have been removed, and the deposit is intended to meet all of the requirements of 37 C.F.R. §1.801-1.809. The NCIMB Accession Number is NCIMB No. 41519. The deposit will be maintained in the depository for a period of 30 years, or 5 years after the last request, or for the effective life of the patent, whichever is longer, and will be replaced as necessary during that period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A Ptilotus exaltatus seed, wherein said seed has a natural germination rate of 61% to 100%.

2. The Ptilotus seed of claim 1, wherein said natural germination rate is between 61.0% and 69.0%.

3. The Ptilotus seed of claim 1, wherein said natural germination rate is between 69.1% and 75%.

4. The Ptilotus seed of claim 1, wherein said natural germination rate is between 75.1% and 80.0%.

5. The Ptilotus seed of claim 1, wherein said natural germination rate is between 80.1% and 85.0%.

6. The Ptilotus seed of claim 1, wherein said natural germination rate is between 85.1% and 90.0%.

7. The Ptilotus seed of claim 1, wherein said natural germination rate is between 90.1% and 95.0%.

8. The Ptilotus seed of claim 1, wherein said natural germination rate is between 95.1% and 100%.

9. The Ptilotus seed of claim 1, representative sample of said seed was deposited under NCIMB No. 41519.

10. A Ptilotus exaltatus plant, wherein said plant has a plant height between 350.0 mm and 450.0 mm when grown without a growth regulator.

11. A seed of the Ptilotus plant of claim 10, representative sample seed of said seed was deposited under NCIMB No. 41519.

12. A Ptilotus exaltatus plant, wherein said plant has a plant height between 250.0 mm and 310.0 mm when grown with using a growth regulator.

13. A seed of the Ptilotus plant of claim 12, representative sample seed of said seed was deposited under NCIMB No. 41519.

14. A Ptilotus exaltatus plant, wherein said plant has a number of days to flower between 75 days and 100 days when grown without using a growth regulator.

15. The Ptilotus plant of claim 14, wherein said number of days to flower is between 75 days and 80 days.

16. The Ptilotus plant of claim 14, wherein said number of days to flower is between 81 days and 90 days.

17. The Ptilotus plant of claim 14, wherein said number of days to flower is between 90 days and 100 days.

18. A seed of the Ptilotus plant of claim 14, representative sample seed of said plant was deposited under NCIMB No. 41519.

19. A Ptilotus exaltatus plant, wherein said plant has a number of days to flower between 80 days and 105 days when grown with a growth regulator.

20. The Ptilotus plant of claim 19, wherein said number of days to flower is between 80 days and 90 days.

21. The Ptilotus plant of claim 19, wherein said number of days to flower is between 91 days and 105 days.

22. A seed of the Ptilotus plant of claim 19, representative sample seed of said plant was deposited under NCIMB No. 41519.
